(19) United States
(12) Patent Application Publication
(10) Pub. No.: US 2025/0177190 A1
(43) Pub. Date: Jun. 5, 2025

(54) FLUID COLLECTION ASSEMBLIES INCLUDING AT LEAST ONE OF A PROTRUSION OR AT LEAST ONE EXPANDABLE MATERIAL

(71) Applicant: PUREWICK CORPORATION, Covington, GA (US)

(72) Inventors: Jill W. Jones, Avondale Estates, GA (US); Christopher D. Drobnik, Wauconda, IL (US); Michael W. Drobnik, Downers Grove, IL (US); Patrick Hudson Chancy, Dunwoody, GA (US); Hoang D. Nguyen, Smyrna, GA (US); Kathleen Davis, Atlanta, GA (US)

(21) Appl. No.: 19/049,783

(22) Filed: Feb. 10, 2025

Related U.S. Application Data
(63) Continuation of application No. 17/501,591, filed on Oct. 14, 2021, now Pat. No. 12,257,174.
(60) Provisional application No. 63/094,608, filed on Oct. 21, 2020.

(51) Int. Cl. A61F 5/455; A61L 26/00

(52) U.S. Cl. CPC A61F 5/455; A61L 26/0009; A61L 26/008; A61L 26/0085

(57) ABSTRACT

An example female fluid collection assembly includes a fluid impermeable barrier. The fluid impermeable barrier at least defines a chamber and an opening. The female fluid collection assembly also includes at least one porous material disposed in the chamber. The porous material extends across the opening. In an embodiment, the portion of the porous material that extends across the opening include a protrusion that extends outwardly from the opening. In an embodiment, the porous material includes at least one expandable material that is configured to expand (e.g., increase in volume) when the expandable material exposed to one or more bodily fluids (e.g., urine).

### CROSS-REFERENCE TO RELATED APPLICATION(S)

This application is a continuation of U.S. application Ser. No. 17/501,591 filed on Oct. 14, 2021, which claims priority to U.S. Provisional Patent Application No. 63/094,608 filed on Oct. 21, 2020, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection assemblies continue to seek new and improved assemblies, systems, and methods to collect urine.

### SUMMARY

Embodiments are directed to fluid collection assemblies that include at least one of a protrusion or at least one expandable material, systems including the same, and method of using the same are disclosed herein. In an embodiment, a fluid collection assembly is disclosed. The fluid collection assembly includes a fluid impermeable barrier at least defining a chamber, at least one opening, and a fluid outlet. The fluid collection assembly also includes at least one porous material disposed in the chamber. The at least one porous material includes at least one of a protrusion extending outwardly from the at least one opening or at least one expandable material that expands when exposed to one or more bodily fluids.

In an embodiment, a fluid collection system is disclosed. The fluid collection system includes a fluid collection assembly. The fluid collection assembly includes a fluid impermeable barrier at least defining a chamber, at least one opening, and a fluid outlet. The fluid collection assembly also includes at least one porous material disposed in the chamber. The at least one porous material includes at least one of a protrusion extending outwardly from the at least one opening or at least one expandable material that expands when exposed to one or more bodily fluids. The fluid collection system also includes fluid storage container and a vacuum source. The chamber of the fluid collection assembly, the fluid storage container, and the vacuum source are in fluid communication with each other via one or more conduits.

In an embodiment, a method of forming a fluid collection assembly is disclosed. The method includes providing at least one porous material. The at least one porous material includes at least one of a protrusion extending outwardly from the at least one opening or at least one expandable material that expands when exposed to one or more bodily fluids. The method also includes disposing the at least one porous material in a chamber defined by a fluid impermeable barrier of the fluid collection, the fluid impermeable barrier defining a fluid outlet.

In an embodiment, a method of using a fluid collection assembly is disclosed. The method includes positioning at least one opening of the fluid collection assembly adjacent to a urethral opening of a patient. The fluid collection assembly includes a fluid impermeable barrier defining a chamber, at least one opening, and at least one fluid outlet. The fluid collection assembly also includes at least one porous material disposed in the chamber. The at least one porous material includes at least one of a protrusion extending outwardly from the at least one opening or at least one expandable material that expands when exposed to one or more bodily fluids. The method also includes receiving one or more bodily fluids from the urethral opening into the chamber.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIGS. 1B and 1C are cross-sectional schematics of the fluid collection assembly taken along planes 1B-1B and 1C-1C, respectively, according to an embodiment.

FIG. 1D is a cross-sectional schematic of the fluid collection assembly after the expandable material is exposed to the bodily fluids, according to an embodiment.

FIG. 2 is a cross-sectional schematic of a fluid collection assembly, according to an embodiment.

FIGS. 3 and 4 are cross-sectional schematics of different fluid collection assemblies that include a fluid permeable support, according to different embodiments.

FIGS. 5-7 are cross-sectional schematics of different fluid collection assemblies that do not include an expandable material, according to different embodiments.

FIG. 8A is an isometric view of a fluid collection assembly, according to an embodiment.

FIG. 8B is a cross-sectional view of the fluid collection assembly of FIG. 8A taken along the plane 8B-8B of FIG. 8A, according to an embodiment.

FIG. 9 is a block diagram of a system for fluid collection, according to an embodiment.

### DETAILED DESCRIPTION

Embodiments are directed to fluid collection assemblies that include at least one of a protrusion or at least one expandable material, systems including the same, and method of using the same are disclosed herein. An example female fluid collection assembly includes a fluid impermeable barrier. The fluid impermeable barrier at least defines a chamber, at least one opening, and a fluid outlet. The female fluid collection assembly also includes at least one porous material disposed in the chamber. The porous material extends across the opening. In an embodiment, the porous material includes at least one expandable material that is configured to expand (e.g., increase in volume) when the expandable material exposed to one or more bodily fluids (e.g., urine). In an embodiment, the portion of the porous material that extends across the opening include a protrusion that extends outwardly from the opening.

During use, the female fluid collection assembly is disposed with the opening of the fluid collection assembly adjacent to a urethral opening of a female patient. The urethral opening may discharge one or more bodily fluids, such a urine, blood, or sweat. The bodily fluids may be received by the porous material and wicked away from the urethral opening and into the chamber. The bodily fluids may be removed from the chamber via a fluid outlet defined by the fluid impermeable barrier. For example, a suction force may be applied to a conduit that is at least partially disposed in the fluid outlet and the suction force may pull the bodily fluids into the conduit and out of the chamber.

Some of the bodily fluids that are discharged from the urethral opening may leak from the chamber. The leaked bodily fluids may cause patient (e.g., an individual using the fluid collection assembly) discomfort, embarrassment, and create unsanitary conditions that require cleaning. Poor fit between the female fluid collection assembly and the region about the urethral opening may cause the bodily fluids to leak from the female fluid collection assembly. For example, the poor fit may cause gaps to be present between the porous material that extends across the opening and the region about the urethral opening. These gaps may provide locations through which the bodily fluids may flow without being received by the porous material and/or locations at which bodily fluids may leave the porous material. The gaps may form between the porous material and the region about the urethral opening for a variety of reasons. For example, variations in the size, shape (e.g., curvature), and anatomy (e.g., labia folds) of the region about the urethral opening may cause gaps to form between the porous material and the region about the urethral opening.

In an embodiment, the female fluid collection assemblies disclosed herein include at least one porous material having at least one expandable material. The also expandable material is configured to increase in volume when the expandable material is exposed to the bodily fluids. The increase in volume of the expandable material may at least one of form a protrusion or cause the porous material to conform to the shape of the region about the urethral opening thereby minimizing or eliminating gaps. Further, conforming the porous material to the shape of the region about the urethral opening may also prevent or at least inhibit movement and shape changes of the female fluid collection assembly. When the expandable material forms a protrusion, the protrusion may be able to be positioned at least partially between the labia folds of the patient which minimizes or reduces gaps formed between the labia folds, between the labia folds and the porous material, and between the urethral opening and the porous material through which bodily fluids may leak. In an embodiment, instead of or in addition to the expandable material, the at least one porous material of the fluid collection assemblies disclosed herein includes a protrusion. The protrusion may minimize or prevent the formation of gaps between the porous material and the region about the urethral opening. For example, the protrusion may be able to be positioned at least partially between the labia folds of the patient which minimizes or reduces gaps formed between the labia folds, between the labia folds and the porous material, and between the urethral opening and the porous material through which bodily fluids may leak. Further, the labia folds may also hold the protrusion such that the position and/or shape of the fluid collection assembly does not move, thereby eliminating gaps caused by the fluid collection assembly moving or changing shape.

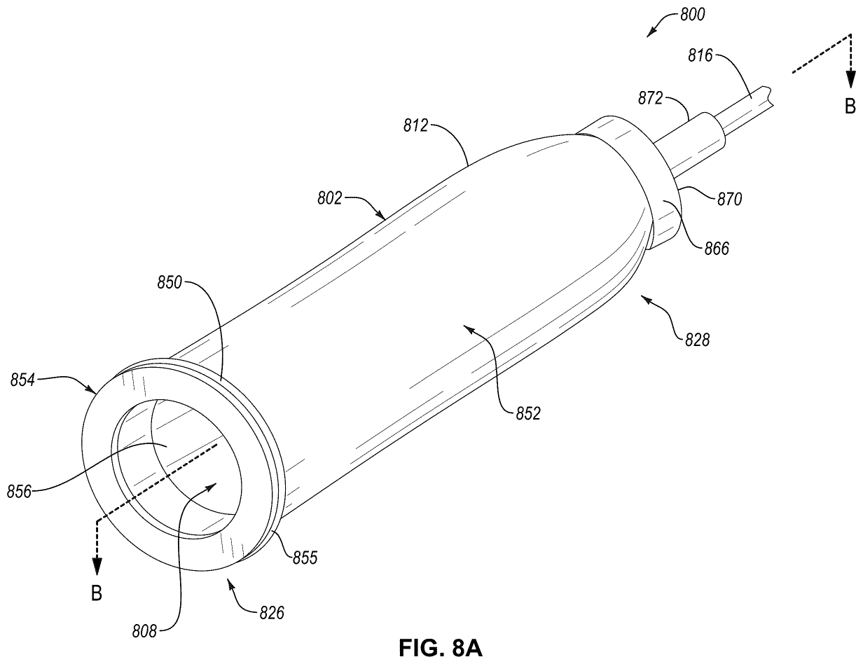

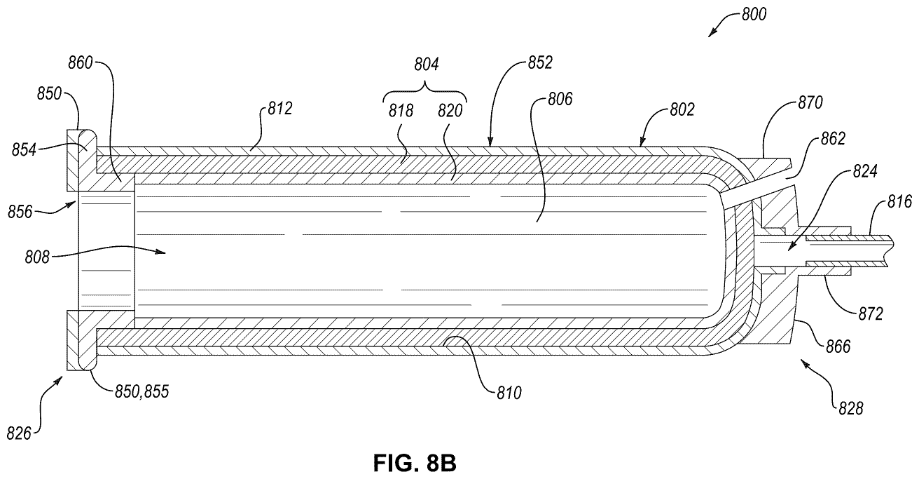

As will be discussed in more detail with regards to FIGS. 8A and 8B, the expandable material may be used in a male fluid collection assembly. The male fluid collection assembly is configured to receive bodily fluids from a male urethral opening (e.g., penis).

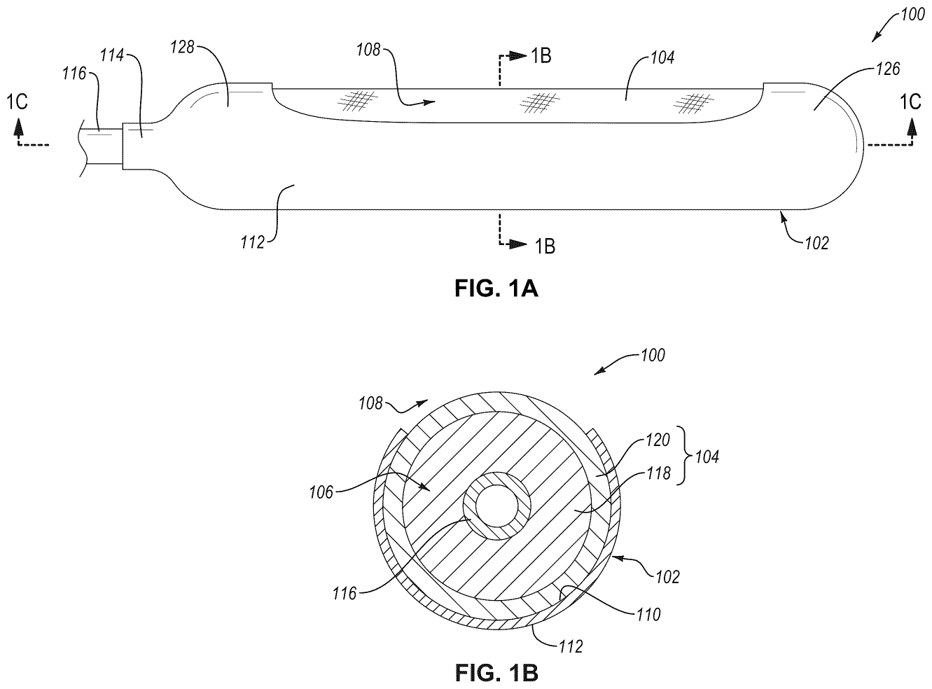

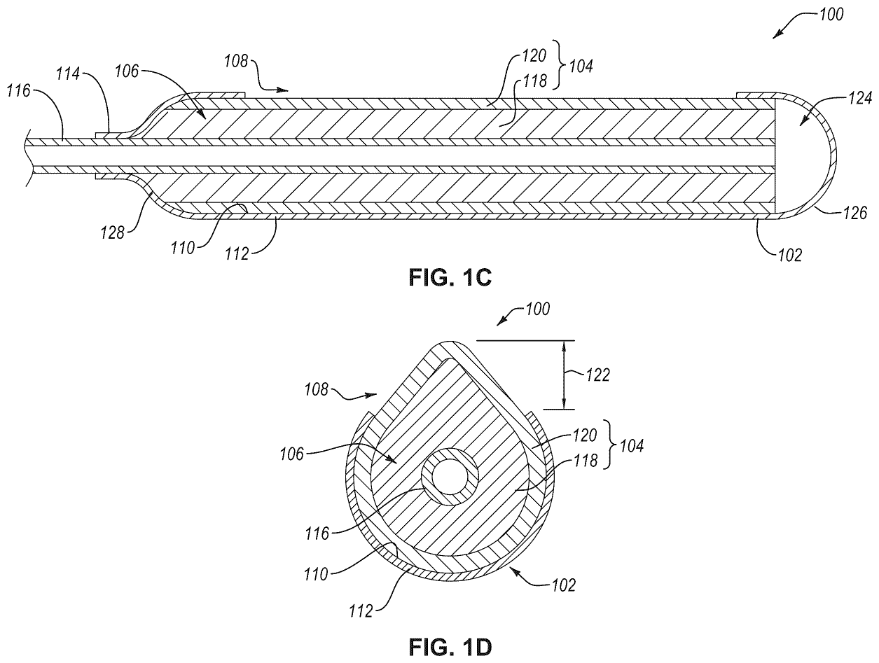

FIG. 1A is an isometric view of a fluid collection assembly 100, according to an embodiment. FIGS. 1B and 1C are cross-sectional schematics of the fluid collection assembly 100 taken along planes 1B-1B and 1C-1C, respectively, according to an embodiment. The fluid collection assembly 100 is an example of a female fluid collection assembly for receiving and collecting one or more bodily fluids from a female. The fluid collection assembly 100 includes a fluid impermeable barrier 102 and at least one porous material 104 disposed in the fluid impermeable barrier 102.

The fluid impermeable barrier 102 at least partially defines a chamber 106 (e.g., interior region) and an opening 108. For example, the interior surface(s) 110 of the fluid impermeable barrier 102 at least partially define the chamber 106 within the fluid collection assembly 100. The fluid impermeable barrier 102 temporarily stores the bodily fluids in the chamber 106. The fluid impermeable barrier 102 may be formed of any suitable fluid impermeable material(s), such as a fluid impermeable polymer (e.g., silicone, polypropylene, polyethylene, polyethylene terephthalate, a polycarbonate, neoprene, etc.), a metal film, natural rubber, another suitable material, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least one or more portions of at least an outer surface 112 of the fluid impermeable barrier 102 may be formed from a soft and/or smooth material, thereby reducing chaffing.

In some examples, the fluid impermeable barrier 102 may be tubular (ignoring the opening), such as substantially cylindrical (as shown), oblong, prismatic, or flattened tubes. During use, the outer surface 112 of the fluid impermeable barrier 102 may contact the patient. The fluid impermeable barrier 102 may be sized and shaped to fit in the gluteal cleft between the legs of a female user.

The opening 108 provides an ingress route for the bodily fluids to enter the chamber 106. The opening 108 may be defined by the fluid impermeable barrier 102, such as by an inner edge of the fluid impermeable barrier 102. For example, the opening 108 is formed in and extends through the fluid impermeable barrier 102, from the outer surface 112 to the inner surface 110, thereby enabling the bodily fluids to enter the chamber 106 from outside of the fluid collection assembly 100. The opening 108 may be an elongated hole in the fluid impermeable barrier 102. For example, the opening 108 may be defined as a cut-out in the fluid impermeable barrier 102. The opening 108 may be located and shaped to be positioned adjacent to a female urethral opening.

The fluid collection assembly 100 may be positioned proximate to the female urethral opening and the bodily fluids may enter the chamber of the fluid collection assembly 100 via the opening 108. The fluid collection assembly 100 is configured to receive the bodily fluids into the chamber 106 via the opening 108. When in use, the opening 108 may have an elongated shape that extends from a first location below the urethral opening (e.g., at or near the anus or the vaginal opening) to a second location above the urethral opening (e.g., at or near the top of the vaginal opening or the mons pubis).

The opening 108 may have an elongated shape because the space between the legs of a female is relatively small when the legs of the female are closed, thereby only permitting the flow of the bodily fluids along a path that corresponds to the elongated shape of the opening 108 (e.g., longitudinally extending opening). The opening 108 in the fluid impermeable barrier 102 may exhibit a length that is measured along the longitudinal axis of the fluid collection assembly 100 that may be at least about 10% of the length of the fluid collection assembly 100, such as about 25% to about 50%, about 40% to about 60%, about 50% to about 75%, about 65% to about 85%, or about 75% to about 95% of the length of the fluid collection assembly 100.

The opening 108 in the fluid impermeable barrier 102 may exhibit a width that is measured transverse to the longitudinal axis of the fluid collection assembly 100 that may be at least about 10% of the circumference of the fluid collection assembly 100, such as about 25% to about 50%, about 40% to about 60%, about 50% to about 75%, about 65% to about 85%, or about 75% to about 100% of the circumference of the fluid collection assembly 100. The opening 108 may exhibit a width that is greater than 50% of the circumference of the fluid collection assembly 100 since the vacuum (e.g., suction) through the conduit 116 pulls the bodily fluids through the porous material 104 and into the conduit 116. In some examples, the opening 108 may be vertically oriented (e.g., having a major axis parallel to the longitudinal axis of the fluid collection assembly 100). In some examples (not shown), the opening 108 may be horizontally oriented (e.g., having a major axis perpendicular to the longitudinal axis of the fluid collection assembly 100). In an example, the fluid impermeable barrier 102 may be configured to be attached to the individual, such as adhesively attached (e.g., with a hydrogel adhesive) to the individual. According to an example, a suitable adhesive is a hydrogel layer.

In some examples, the fluid impermeable barrier 102 may define a fluid outlet 114 sized to receive the conduit 116. The at least one conduit 116 may be disposed in the chamber 106 via the fluid outlet 114. The fluid outlet 114 may be sized and shaped to form an at least substantially fluid tight seal against the conduit 116 or the at least one tube thereby substantially preventing the bodily fluids from escaping the chamber 106.

The fluid impermeable barrier 102 may include markings thereon, such as one or more markings to aid a user in aligning the fluid collection assembly 100 on the patient. For example, a line on the fluid impermeable barrier 102 (e.g., opposite the opening 108) may allow a healthcare professional to align the opening 108 over the urethral opening of the patient. In examples, the markings may include one or more of alignment guide or an orientation indicator, such as a stripe or hashes. Such markings may be positioned to align the fluid collection assembly 100 to one or more anatomical features such as a pubic bone, etc.

As previously discussed, the fluid collection assembly 100 includes the porous material 104 disposed in the chamber 106. The porous material 104 may cover at least a portion (e.g., all) of the opening 108. The porous material 104 is exposed to the environment outside of the chamber 106 through the opening 108. In an embodiment, as illustrated, the porous material 104 include at least one expandable material 118 and a fluid permeable membrane 120. However, as will be discussed in more detail below, at least one of the expandable material 118 or the fluid permeable membrane 120 may be omitted from the porous material 104. Further, the porous material 104 may include a fluid permeable support (FIGS. 3-6) instead of or in addition to at least one of the expandable material 118 and the fluid permeable membrane 120.

As previously discussed, the expandable material 118 is configured to increase in volume when the expandable material 118 is exposed to the bodily fluids. For example, the expandable material 118 may include at least one of one or more absorbent materials, one or more adsorbent materials, or one or more hydrophilic materials. Specific examples of the expandable material 118 include hydrogels, super absorbent polymers (e.g., polymers that absorb at least 10 times its weight in bodily fluids, at least 25 times its weight in bodily fluids, at least 50 times its own weight in bodily fluids, at least 100 times its own weight in bodily fluids, or at least 200 times its own weight in bodily fluids), or polymeric sponges. In an embodiment, such expandable materials 118 may retain at least some of the bodily fluids that are received thereby, such as water that is contained within the bodily fluids. The bodily fluids that are retained by the expandable material 118 may increase the volume of the expandable material 118.

FIG. 1B illustrates the expandable material 118 before the expandable material 118 is exposed to the bodily fluids (e.g., the expandable material 118 is dry). In other words, FIG. 1B illustrates the expandable material 118 when the expandable material 118 exhibits a first, initial volume. The expandable material 118 may exhibit a first, initial shape, when the expandable material 118 exhibits the first volume. In the illustrated embodiment, the first shape may exhibit a generally circular cross-sectional shape (e.g., a generally cylindrical shape). However, the initial shape may exhibit a generally triangular cross-sectional shape, a generally trapezoidal cross-sectional shape, a rectangular (e.g., square) cross-sectional shape, an oblong cross-sectional shape, a tear-drop cross-sectional shape, or any other suitable shape. The first shape may generally correspond to the shape of the fluid impermeable barrier 102 and may not include a protrusion.

Exposing the expandable material to the bodily fluids causes the expandable material 118 to expand thereby at least increasing the volume of the expandable material 118. For example, FIG. 1D is a cross-sectional schematic of the fluid collection assembly 100 after the expandable material 118 is exposed to the bodily fluids, according to an embodiment. As illustrated, the expandable material 118 exhibits a second volume after the expandable material 118 is exposed to the bodily fluids. The second volume of the expandable material 118 is greater than the first volume.

In an embodiment, as illustrated, exposing the expandable material 118 to the bodily fluids causes the expandable material 118 to change a shape thereof. For example, the increased volume of the expandable material 118 causes the expandable material 118 to apply a force to the fluid impermeable barrier 102. When the fluid impermeable barrier 102 is formed from a relatively flexible material (e.g., silicone, neoprene, etc.), the force applied from the expandable material 118 to the fluid impermeable barrier 102 may cause the fluid impermeable barrier 102 to change a shape thereof (e.g., bow outwardly) and/or stretch. When the fluid impermeable barrier 102 is formed from a relatively stiff material (e.g., a metal), the force applied from the expandable material 118 to the fluid impermeable barrier 102 substantially does not change a shape of the fluid impermeable barrier 102. Regardless if the fluid impermeable barrier 102 is formed from a relatively flexible or relatively rigid material, the fluid impermeable barrier 102 applies a force normal to the force applied from the expandable material 118 to the fluid impermeable barrier 102. The force applied from the fluid impermeable barrier 102 to the expandable material 118 limits the volume increase of portions of the expandable material 118 adjacent to the fluid impermeable barrier 102. However, no force is directly applied from the fluid impermeable barrier 102 to the portions of the expandable material 118 adjacent to the opening 108. Thus, the volume of the portions of the expandable material 118 adjacent to the opening 108 may more freely expand than portions of the expandable material 118 adjacent to the fluid impermeable barrier 102. The difference in the expansion in portions of the expandable material 118 adjacent to the fluid impermeable barrier 102 and adjacent to the opening 108 causes the shape of the expandable material 118 to change (e.g., from a first shape to a second shape). For example, the greater expansion of the portions of the expandable material 118 adjacent to the opening 108 causes the expandable material 118 to protrude outwardly from the opening 108 to form a protrusion 122.

The protrusion 122 may exhibit a generally triangular cross-sectional shape, a generally at least partially semi-circular cross-sectional shape, or any other suitable cross-sectional shape when the protrusion 122 does not abut the region about the urethral opening of the patient. When the protrusion 122 exhibits a generally triangular cross-sectional shape, the protrusion 122 may be facilitate insertion of the protrusion 122 between the labia folds of the patient. For example, the generally triangular cross-sectional shape of the protrusion 122 may form a wedge that may separate and move between the labia folds thereby positioning the protrusion 122 adjacent to the urethral opening of the patient. In a particular embodiment, the porous material 104 (e.g., the expandable material 118) and the fluid collection assembly 100 may exhibit a generally tear-drop shape (e.g., a generally circular cross-sectional shape with a generally triangular cross-sectional shape extending therefrom) when the protrusion 122 exhibits the generally triangular cross-sectional shape. For example, the fluid collection assembly 100 may exhibit a generally circular cross-sectional shape when the expandable material 118 exhibits the first shape. This allows the fluid collection assembly 100 to include a generally cylindrical fluid impermeable barrier 102, as used in some convention fluid collection assemblies. Using a generally cylindrical fluid impermeable barrier 102 allows the use of fluid impermeable barriers that are readily available and does not require the use of specialty parts. In other words, using a generally cylindrical fluid impermeable barrier 102 may prevent logistical issues (e.g., lack of supply) caused by using non-cylindrical fluid impermeable barriers. The generally cylindrical fluid impermeable barriers 102 may be formed from silicone, neoprene, or other relatively flexible polymers. Even though the generally cylindrical fluid impermeable barrier 102 is formed from such relatively flexible materials, the generally cylindrical fluid impermeable barrier 102 may retain the generally circular cross-sectional shape thereof when the volume of the expandable material 118 increases. This may cause the expandable material 118 to extend from the opening 108 to form a protrusion 122 exhibiting the generally triangular cross-sectional shape. In other words, forming the fluid impermeable barrier 102 from readily available generally cylindrical fluid impermeable barriers may cause the fluid collection assembly 100 to exhibit the generally tear-drop shape.

The shape of the expandable material 118 may conform to the shape of the region about the urethral opening of the patient when the porous material 104 comes in contact with the region about the urethral opening. For example, the portions of the expandable material 118 that form the protrusion 122 may press, directly or indirectly, against the region about the urethral opening when the volume of the expandable material 118 increases. Similar to the fluid impermeable barrier 102, the region about the urethral opening applies a force normal to the force that the expandable material 118 applies to the region about the urethral opening which restricts volume increases of the expandable material 118. However, at least initially, only a portion of the region about urethral opening contacts the porous material 104. Thus, at least initially, the region about the urethral opening only restricts the volume increase of the portion of the expandable material 118 that contacts (directly or indirectly) the region about the urethral opening. The portions of the expandable material 118 that does not contact the region about the urethral opening substantially do not have the volume increases thereof restricted which allows such portions the expandable material 118 to expand into gaps between the porous material 104 and the region about the urethral opening. Thus, the expandable material 118, and in particular the portions of the expandable material 118 that form the protrusion 122, conform to the region about the urethral opening and prevent the formation of gaps.

As previously discussed, the fluid collection assembly 100 may include the fluid permeable membrane 120 disposed in the chamber 106. The fluid permeable membrane 120 may cover at least a portion (e.g., all) of the opening 108.

In an embodiment, the fluid permeable membrane 120 is exposed to the environment outside of the chamber 106 through the opening 108. The fluid permeable membrane 120 may be configured to wick any bodily fluids away from the opening 108, thereby preventing the fluid from escaping the chamber 106. The permeable properties referred to herein may be wicking, capillary action, diffusion, or other similar properties or processes, and are referred to herein as “permeable” and/or “wicking.” Such “wicking” may not include absorption of fluid into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the wicking material (e.g., absorbency), such as less than about 30 wt % of the dry weight of the wicking material, less than about 20 wt %, less than about 15 wt %, less than about 10 wt %, less than about 7 wt %, less than about 5 wt %, less than about 3 wt %, less than about 2 wt %, less than about 1 wt %, or less than about 0.5 wt % of the dry weight of the wicking material. Thus, the fluid permeable membrane 120 may remove the bodily fluids away from the region about the urethral opening and inhibit bodily fluids that are present in the expandable material 118 from back flowing to the region about the urethral opening. In other words, the fluid permeable membrane 120 may maintain the region about the urethral opening dry when the fluid permeable membrane 120 is configured to wick the bodily fluids away from the opening 108. The fluid permeable membrane 120 may also wick the fluid generally towards an interior of the chamber 106, as discussed in more detail below.

The fluid permeable membrane 120 may include any material that may wick the bodily fluids. For example, the fluid permeable membrane 120 may include fabric, such as a gauze (e.g., a silk, linen, or cotton gauze), another soft fabric, or another smooth fabric. Forming the fluid permeable membrane 120 from gauze, soft fabric, and/or smooth fabric may reduce chaffing caused by the fluid collection assembly 100. In an example, the fluid permeable membrane 120 may include spun nylon fibers. In an embodiment, the fluid permeable membrane 120 may include at least one absorbent or adsorbent material instead of or in addition to at least one wicking material.

In an embodiment, the fluid permeable membrane 120 may provide structure to the porous material 104. For example, the expandable material 118 may be formed from a material that has no structure. As used herein, “no structure” refers to a material that, in bulk, is substantially unable to resist shear forces. Examples of expandable materials 118 that have no structure include some hydrogels, some super absorbent polymers, powders, and beads. Expandable materials 118 that have no structure may be unable to remain in the chamber 106 when the porous material 104 only includes such expandable materials. However, the fluid permeable membrane 120 may at least partially enclose expandable material 118. Thus, the fluid permeable membrane 120 may maintain the expandable materials 118 that have no structure in the chamber 106.

In an embodiment, the fluid permeable membrane 120 is sufficiently flexible (e.g., the fluid permeable membrane 120 is formed from certain gauzes) that the minimal force applied from the fluid permeable membrane 120 to the expandable material 118 is at most about 50% (e.g., at most about 40%, at most about 30%, at most about 25%, at most about 20%, at most about 15%, at most about 10%, at most about 7.5%, at most about 5%, at most about 2.5%, or at most about 1%) the force applied to the expandable material 118 from the fluid impermeable barrier 102 or the region about the urethral opening of the patient during normal use. For example, the fluid permeable membrane 120 may applied a normal force of 5 N or less to the expandable material 118 if the fluid impermeable barrier 102 applies a normal force of 10 N to the expandable material 118. As such, in such an embodiment, the shape of the expandable material 118 is more controlled by the fluid impermeable barrier 102 and, more particularly, the region about the urethral opening of the patient than the fluid permeable membrane 120 which allows the expandable material 118 to exhibit a shape that corresponds to the shape of the region about the urethral opening when the expandable material 118 expands (e.g., receives the bodily fluids). In other words, the fluid permeable membrane 120 substantially does not prevent the expandable material 118 from conforming to the shape of the region about the urethral opening of the patient.

In an embodiment, the fluid permeable membrane 120 is sufficiently rigid that the minimal normal force applied from the fluid permeable membrane 120 to the expandable material 118 is 50% or greater the force applied to the expandable material 118 from the fluid impermeable barrier 102 or the region about the urethral opening of the patient during normal use. In such an embodiment, the fluid permeable membrane 120 may limit the shape of the expandable material 118 may take as the volume of the expandable material 118 increases. For example, the fluid permeable membrane 120 may exhibit such rigidity when the fluid permeable membrane 120 exhibits a Young's modulus and/or percent elongation that with 10% or greater than the fluid impermeable barrier 102. Limiting the shape of the expandable material 118 with the fluid permeable membrane 120 may prevent over expansion of the expandable material 118 which may push the fluid impermeable barrier 102 and the chamber 106 away from the patient and prevent oversaturation of the expandable material 118, either of which may promote leaks of the bodily fluids.

Regardless if the fluid permeable membrane 120 is configured to apply a force to the expandable material 118 that is less than or greater than 50% the force applied from the fluid impermeable barrier 102 and the region about the urethral opening during normal use, the fluid permeable membrane 120 may at least partially control the shape of the protrusion 122. For example, before the protrusion 122 contacts the urethral opening, the fluid permeable membrane 120 may apply a force to the expandable material 118 that controls the shape of the protrusion 122. In an embodiment, the fluid permeable membrane 120 may be configured to cause the protrusion 122 to exhibit a generally triangular shape.

The porous material 104 may at least substantially completely fill the portions of the chamber 106 that are not occupied by the conduit 116. In some examples, the porous material 104 may not substantially completely fill the portions of the chamber 106 that are not occupied by the conduit 116. In such an example, the fluid collection assembly 100 includes the fluid reservoir 124 (FIG. 1C) disposed in the chamber 106.

The fluid reservoir 124 is a substantially unoccupied portion of the chamber 106. The fluid reservoir 124 may be defined between the fluid impermeable barrier 102 and porous material 104. The bodily fluids that are in the chamber 106 may flow through the porous material 104 to the fluid reservoir 124. The fluid reservoir 124 may retain of the bodily fluids therein.

The bodily fluids that are in the chamber 106 may flow through the porous material 104 to the fluid reservoir 124. The fluid impermeable barrier 102 may retain the bodily fluids in the fluid reservoir 124. While depicted in the distal end region 126, the fluid reservoir 124 may be located in any portion of the chamber 106 such as the proximal end region 128. The fluid reservoir 124 may be located in a portion of the chamber 106 that is designed to be located in a gravimetrically low point of the fluid collection assembly 100 when the fluid collection assembly 100 is worn.

In some examples (not shown), the fluid collection assembly 100 may include multiple reservoirs, such as a first reservoir that is located at the portion of the chamber 106 at the distal end region 126 and a second reservoir that is located at the portion of the of the chamber 106 that at the proximal end region 128. In another example, the porous material 104 is spaced from at least a portion of the conduit, and the fluid reservoir 124 may be the space between the porous material 104 and the conduit 116.

The conduit 116 may be at least partially disposed in the chamber 106. The conduit 116 may be used to remove the bodily fluids from the chamber 106. The conduit 116 (e.g., a tube) includes an inlet and an outlet positioned downstream from the inlet. The outlet may be operably coupled to a suction source, such as a vacuum pump for withdrawing fluid form the chamber through the conduit 116. For example, the conduit 116 may extend into the fluid impermeable barrier 102 from the proximal end region 128 and may extend to the distal end region 126 to a point proximate to the fluid reservoir 124 therein such that the inlet is in fluid communication with the fluid reservoir 124. The conduit 116 fluidly couples the chamber 106 with the fluid storage container (not shown) or the vacuum source (not shown).

The conduit 116 may include a flexible material such as plastic tubing (e.g., medical tubing). Such plastic tubing may include a thermoplastic elastomer, polyvinyl chloride, ethylene vinyl acetate, polytetrafluorocthylene, etc., tubing. In some examples, the conduit 116 may include silicon or latex. In some examples, the conduit 116 may include one or more portions that are resilient, such as to by having one or more of a diameter or wall thickness that allows the conduit to be flexible.

As shown in FIG. 1C, the end of the conduit 116 may extend through a bore in the porous material 104, such as into or adjacent to the fluid reservoir 124. For example, the inlet of the conduit 116 may be extend into or be positioned in the fluid reservoir 124. In some examples, the inlet may not extend into the fluid reservoir 124. In such examples, the inlet may be disposed within the porous material 104 or at a terminal end thereof. For example, an end of the conduit 116 may be coextensive with or recessed within the porous material 104.

Locating the inlet of the conduit 116 at or near a location expected to be the gravimetrically low point of the chamber 106 when worn by a user enables the conduit 116 to receive more of the bodily fluids than if inlet of the conduit 116 was located elsewhere and reduce the likelihood of pooling (e.g., pooling of the bodily fluids may cause microbe growth and foul odors). For instance, the bodily fluids in the porous material 104 may flow in any direction due to capillary forces. However, the bodily fluids may exhibit a preference to flow in the direction of gravity, especially when at least a portion of the porous material 104 is saturated with the bodily fluids. Accordingly, one or more of the inlet of the conduit 116 or the fluid reservoir 124 may be located in the fluid collection assembly 100 in a position expected to be the gravimetrically low point in the fluid collection assembly 100 when worn by a patient, such as the distal end region 126.

In an example, the conduit 116 is configured to be at least insertable into the chamber 106. In such an example, the conduit 116 may include one or more markers (not shown) on an exterior thereof that are located to facilitate insertion of the conduit 116 into the chamber 106. For example, the conduit 116 may include one or more markings thereon that are configured to prevent over or under insertion of the conduit 116, such as when the conduit 116 defines an inlet that is configured to be disposed in or adjacent to the fluid reservoir 124. In another example, the conduit 116 may include one or more markings thereon that are configured to facilitate correct rotation of the conduit 116 relative to the chamber 106. The one or more markings may include a line, a dot, a sticker, or any other suitable marking.

As described in more detail below, the conduit 116 is configured to be coupled to, and at least partially extend between, one or more of the fluid storage container (not shown) and the vacuum source (not shown). In an example, the conduit 116 is configured to be directly connected to the vacuum source. In such an example, the conduit 116 may extend from the fluid impermeable barrier 102 by at least one foot, at least two feet, at least three feet, or at least six feet. In another example, the conduit 116 is configured to be indirectly connected to at least one of the fluid storage container (not shown) and the vacuum source (not shown). In some examples, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

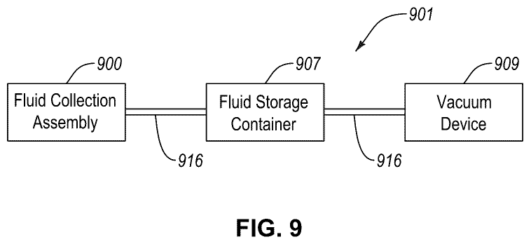

The inlet and the outlet of the conduit 116 are configured to fluidly couple (e.g., directly or indirectly) the vacuum source (not shown) to the chamber 106 (e.g., the fluid reservoir 124). As the vacuum source (FIG. 9) applies a vacuum/suction in the conduit 116, the bodily fluids in the chamber 106 (e.g., in the fluid reservoir 124) may be drawn into the inlet of the conduit 116 and out of the fluid collection assembly 100 via the conduit 116. In some examples, the conduit 116 may be frosted or opaque (e.g., black) to obscure visibility of the bodily fluids therein.

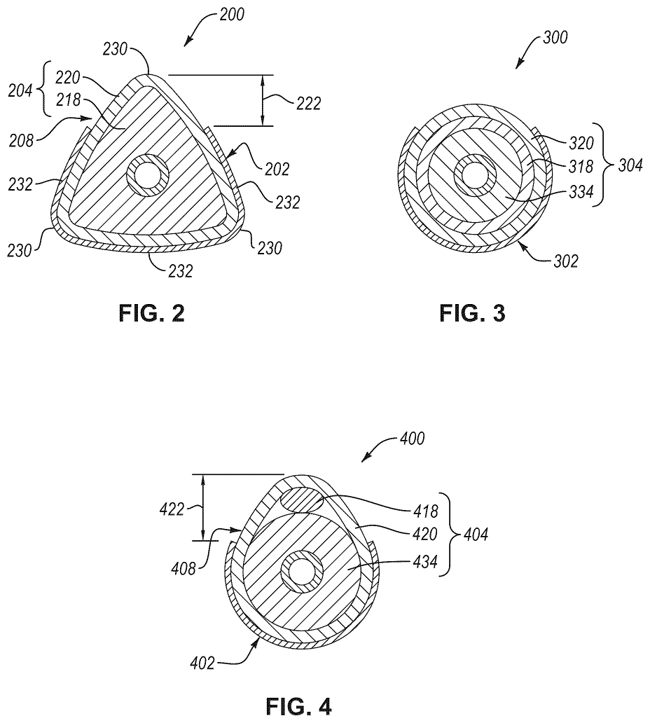

The fluid collection assemblies disclosed herein may exhibit different cross-sectional shapes that the cross-sectional shapes shown in FIGS. 1B and 1D. For example, FIG. 2 is a cross-sectional schematic of a fluid collection assembly 200, according to an embodiment. The fluid collection assembly 200 is an example of a female fluid collection assembly for receiving and collecting one or more bodily fluids from a female. Except as otherwise disclosed herein, the fluid collection assembly 200 may be the same or substantially similar to any of the fluid collection assemblies disclosed herein. For example, the fluid collection assembly 200 may include a fluid impermeable barrier 202 and at least one porous material 204. Also, except as otherwise disclosed herein, the features discussed with regards to FIG. 2 may be used in any of the fluid collection assemblies disclosed herein.

At least a portion of the fluid collection assembly 200 exhibits a generally triangular cross-sectional shape. The fluid collection assembly 200 exhibit a generally triangular cross-sectional shape when the fluid collection assembly 200 form three apex 230 and three edges 232 (e.g., straight or curved edges) extending between adjacent edges 232. The fluid impermeable barrier 202 (ignoring the opening 208) and the porous material 204 (e.g., the expandable material 218 and the fluid permeable membrane 220) may also exhibit a generally triangular cross-sectional shape. The generally triangular cross-sectional shape of the fluid collection assembly 200 may cause the protrusion 222 formed by the porous material 204 to exhibit a generally triangular cross-sectional shape. Further, the apexes 230 that do not form the protrusion 222 may be more likely to contact the thighs of the patient than if the fluid collection assembly 200 exhibited a generally cylindrical shape. In some embodiments, the contact between the fluid impermeable barrier 202 and the thighs of the patient may be used to maintain the shape and position of the fluid collection assembly 200. As such, the generally triangular shape of the fluid collection assembly 200 may help maintain the shape and position of the fluid collection assembly 200.

In an embodiment, the fluid collection assembly 200 may exhibit the generally triangular cross-sectional shape before the expandable material 218 is exposed to the one or more bodily fluids. In such an embodiment, the fluid impermeable barrier 202 and the porous material 204 are provided while exhibiting the generally triangular cross-sectional shape. The generally triangular shape of the protrusion 222 may facilitate positioning the protrusion 222 between the labia folds of the patient before the urethral opening discharges the bodily fluids. The expandable material 218 may then expand when the expandable material 218 is exposed to the bodily fluids such that the protrusion 222 increases in volume. The increase in volume of the protrusion 222 may include at least one of increasing the distance the protrusion 222 extends from the opening 208 or causing the protrusion 222 to fill any gaps remaining after positioning the protrusion 222 between the labia folds. It is noted that the fluid collection assembly 200 may be provided while the fluid collection assembly 200 exhibits a generally tear-drop cross-sectional shape (e.g., the porous material 204 exhibits a generally tear-drop cross-sectional shape) or any of the other cross-sectional shapes disclosed herein.

In an embodiment, the fluid collection assembly 200 may exhibit the generally triangular cross-sectional shape only after the expandable material 218 is exposed to the one or more bodily fluids. In an example, at least a portion of the fluid collection assembly 200 is provided exhibiting a generally trapezoidal shape. In such an example, the fluid impermeable barrier 202 may be provided while exhibiting a generally trapezoidal cross-sectional shape or, ignoring the opening 208, a generally triangular cross-sectional shape. Also, the porous material 204 does not initially include the protrusion 222 or the protrusion 222 does not exhibit a generally triangular shape. Instead, the porous material 204 may at least one of extend across the opening 208 in a generally planar manner, extend across the opening 208 in a generally concave manner, or extend outwardly from the opening 208 in a generally non-triangular manner. The expandable material 218 may be exposed to the one or more bodily fluids and expand such that the fluid collection assembly 200 exhibits the generally triangular cross-sectional shape illustrated in FIG. 2. Alternatively, in an example, at least a portion of the fluid collection assembly 200 may be provided while exhibiting a generally circular cross-sectional shape, as shown in FIG. 1B. In such an example, the fluid impermeable barrier 202 (ignoring the opening 208) and the porous material 204 may exhibit a generally circular cross-sectional shape. When the expandable material 218 is exposed to the bodily fluids, the expandable material 218 expands to form the protrusion 222. The expandable material 218 may also press against the fluid impermeable barrier 202 which causes the fluid impermeable barrier 202 to expand and change shape. For instance, as illustrated, the expandable material 218 may cause the fluid impermeable barrier 202 to change from the generally circular cross-sectional shape to the generally triangular cross-sectional shape. The shape change of the fluid impermeable barrier 202 may be controlled by selectively thinning the portion of the fluid impermeable barrier 202 that form the apexes 230 and/or selectively forming the portions of the fluid impermeable barrier 202 that form the apexes 230 from a material that is more flexible (e.g., exhibits a lower Young's modulus) than the rest of the fluid impermeable barrier 202.

As previously discussed, the porous material used in any of the fluid collection assemblies disclosed herein may include a fluid permeable support in addition to the expandable material and, optionally, the permeable membrane. FIGS. 3 and 4 are cross-sectional schematics of different fluid collection assemblies that include a fluid permeable support, according to different embodiments. The fluid collection assemblies illustrated in FIGS. 3 and 4 are examples of female fluid collection assemblies for receiving and collecting one or more bodily fluids from a female. Except as otherwise disclosed herein, the fluid collection assemblies illustrated in FIGS. 3 and 4 are the same or substantially similar to any of the fluid collection assemblies disclosed herein. Further, the features discussed in relation to FIGS. 3 and 4 may be used in any of the fluid collection assemblies disclosed herein.

Referring to FIG. 3, the fluid collection assembly 300 includes a fluid impermeable barrier 302 defining a chamber 306 and at least one porous material 304 disposed in the chamber 306. The porous material 304 includes at least one expandable material 318 and a fluid permeable membrane 320. It is noted that the expandable material 318 and/or the fluid permeable membrane 320 may be omitted from the porous material 304. The porous material 304 may also include a fluid permeable support 334. In an embodiment, the fluid permeable support 334 is configured to support the expandable material 318 and the fluid permeable membrane 320 since expandable material 318 and/or the fluid permeable membrane 320 may be formed from a relatively foldable, flimsy, or otherwise easily deformable material. For example, the fluid permeable support 334 may be positioned such that the expandable material 318 and the fluid permeable membrane 320 are disposed between the fluid permeable support 334 and the fluid impermeable barrier 302. As such, the fluid permeable support 334 may support and maintain the position of the expandable material 318 and the fluid permeable membrane 320.

In an embodiment, the fluid permeable support 334 may include any material that may wick the bodily fluids (e.g., substantially no absorption of fluid into the material may take place after the material is exposed to the fluid and removed from the fluid for a time). Thus, the fluid permeable support 334 may remove the bodily fluids away from the region about the urethral opening and inhibit bodily fluids from back flowing to the region about the urethral opening. In other words, the fluid permeable support 334 may maintain the region about the urethral opening dry. The fluid permeable support 334 may also wick the fluid generally towards an interior of the chamber 306 For example, the expandable material 318 may retain at least some of the bodily fluids therein. However, the wicking properties of the fluid permeable support 334 may induce at least some of the bodily fluids to flow from the expandable material 318 to the fluid permeable support 334, especially when the expandable material 318 is saturated by bodily fluids. The bodily fluids present in the fluid permeable support 334 may flow towards the conduit 316 due to the wicking properties thereof and, thus, the fluid permeable support 334 may prevent the bodily fluids from leaking. The fluid permeable support 334 may be formed from any of the wicking materials disclosed herein, such as any of the fluid permeable membrane materials disclosed herein above. For example, the fluid permeable membrane material(s) may be utilized in a more dense or rigid form than in the fluid permeable membrane 320 when used as the fluid permeable support 334. The fluid permeable support 334 may be formed from any fluid permeable material that is less deformable than the fluid permeable membrane 320. For example, the fluid permeable support 334 may include a porous polymer (e.g., nylon, polyester, polyurethane, polyethylene, polypropylene, etc.) structure or an open cell foam. For instance, the fluid permeable support 334 may be formed from spun nylon fibers. In some examples, the fluid permeable support 334 may be formed from a natural material, such as cotton, wool, silk, or combinations thereof. In such examples, the material may have a coating to prevent or limit absorption of fluid into the material, such as a water repellent coating. In some examples, the fluid permeable support 334 may be formed from fabric, felt, gauze, or combinations thereof. In an embodiment, the fluid permeable support 334 may include any material that absorbs or adsorbs the bodily fluids.

The fluid permeable support 334 may have a greater ability to wick fluids than the fluid permeable membrane 320, such as to move the fluid inwardly from the outer surface of the fluid collection assembly 300. In some examples, the wicking ability of the fluid permeable support 334 and the fluid permeable membrane 320 may be substantially the same.

The expandable material 318, the fluid permeable membrane 320, and the fluid permeable support 334 may be arranged generally concentrically. For example, the fluid permeable support 334 may form an inner layer, the fluid permeable membrane 320 may form an outer layer that extends generally concentrically relative to the fluid permeable support 334, and the expandable material 318 may be positioned between and concentrically relative to the fluid permeable membrane 320 and the fluid permeable support 334. However, it is noted that the expandable material, the fluid permeable membrane, and the fluid permeable support may exhibit other configurations.

Referring to FIG. 4, the fluid collection assembly 400 includes a fluid impermeable barrier 402 and at least one porous material 404 disposed in the fluid impermeable barrier 402. The porous material 404 includes at least one expandable material 418, a fluid permeable membrane 420, and a fluid permeable support 434. The fluid permeable support 434 may form an inner layer of the porous material 404 thereby allowing the fluid permeable support 434 to support the expandable material 418 and the fluid permeable membrane 420. The fluid permeable membrane 420 may form an outer layer of the porous material 404 thereby allowing the permeable membrane 420 to directly contact the skin of the patient. The expandable material 418 may be positioned between a portion of the fluid permeable membrane 420 and a corresponding portion of the fluid permeable support 434. In other words, the expandable material 418 does not extends concentrically relative to the fluid permeable membrane 420 and the fluid permeable support 434.

The expandable material 418 may be in the form of a rod that is positioned between the fluid permeable membrane 420 and the fluid permeable support 434. The expandable material 418 is or includes a rod when the expandable material 418 extends longitudinally and is not concentric relative to the fluid permeable membrane 420 or the fluid permeable support 434. In an example, when the expandable material 418 is a rod, the expandable material 418 may exhibit a generally circular cross-sectional shape, a generally oblong cross-sectional shape (as shown), a generally crescent cross-sectional shape, a generally rectangular (e.g., square) cross-sectional shape, or any other suitable cross-sectional shape.

Forming the expandable material 418 as a rod allows the expandable material 418 to be easily positioned between the fluid permeable membrane 420 and the fluid permeable support 434. In an example, some conventional porous materials include a fluid permeable membrane disposed concentrically around the fluid permeable support. The rod-shape expandable material 418 may be inserted between the fluid permeable membrane and the fluid permeable support of such conventional porous materials to form the porous material 404 shown in FIG. 4. In an example, the expandable material 418 may be co-extruded with the fluid permeable membrane 420 and the fluid permeable support 434 when the expandable material 418 is a rod. In an embodiment, the expandable material 418 may include a plurality of distinct portions (e.g., distinct beads) that are disposed between the fluid permeable membrane 420 and the fluid permeable support 434.

In an embodiment, the expandable material 418 is disposed between a portion of the fluid permeable membrane 420 and a corresponding portion of the fluid permeable support 434 that are adjacent to the opening 408. In such an embodiment, the expandable material 418 may cause the porous material 404 to form a protrusion 422 before the expandable material 418 is exposed to the bodily fluids. When the fluid impermeable barrier 402 exhibits a generally circular cross-sectional shape, ignoring the opening 408, the expandable material 418 may cause the fluid collection assembly 400 to exhibit a generally tear-drop cross-sectional shape. Further, positioning the expandable material 418 adjacent to the opening 408 may cause the expandable material 418 to not press against or minimize a magnitude that the expandable material 418 presses against the fluid impermeable barrier 402 as the expandable material 418 increases in volume. As such, the force applied from the fluid impermeable barrier 402 to the expandable material 418 is minimized or non-existent.

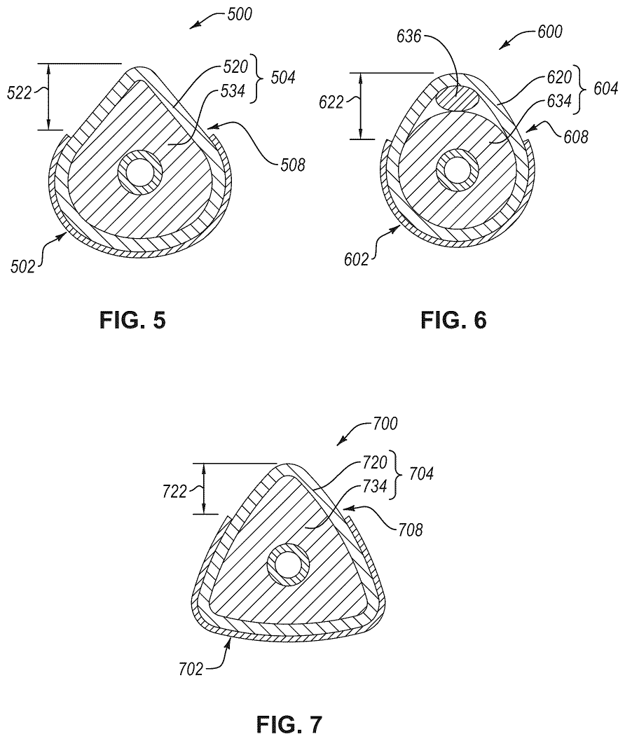

As previously discussed, the expandable material may be omitted from the porous materials disclosed herein. FIGS. 5-7 are cross-sectional schematics of different fluid collection assemblies that do not include an expandable material, according to different embodiments. The fluid collection assemblies illustrated in FIGS. 5-7 are examples of female fluid collection assemblies for receiving and collecting one or more bodily fluids from a female. Except as otherwise disclosed herein, the fluid collection assemblies of FIGS. 5-7 may be the same or substantially similar to any of the fluid collection assemblies disclosed herein. Further, the principles discussed in relation to FIGS. 5-7 may be used in any of the fluid collection assemblies disclosed herein.

Referring to FIG. 5, the fluid collection assembly 500 includes a fluid impermeable barrier 502 and at least one porous material 504 disposed in the fluid impermeable barrier 502. The porous material 504 may include a fluid permeable membrane 520 and a fluid permeable support 534. However, as previously discussed, one of the fluid permeable membrane 520 or the fluid permeable support 534 may be omitted from the fluid collection assembly 500.

The fluid collection assembly 500 may exhibit a generally tear-drop cross-sectional shape. For example, the fluid impermeable barrier 502, ignoring the opening 508, may exhibit a generally circular cross-sectional shape. The portion of the porous material 504 that is disposed in the chamber 506 defined by the fluid impermeable barrier 502 may also exhibit a generally circular cross-sectional shape. The porous material 504 includes a protrusion 522 extending outwardly from the opening 508. The protrusion 522 may exhibit a generally triangular cross-sectional shape.

In an embodiment, the porous material 504 exhibits the generally tear-drop cross-sectional shape without having one or more external forces applied thereto. In an embodiment, the porous material 504 may be molded (e.g., one or more external forces are applied thereto) to exhibit the generally tear-drop shape. For example, the porous material 504 may be provided while exhibiting a generally circular cross-sectional shape. The portion of the porous material 504 may be pitched (i.e., one or more external forces are applied thereto) to form the protrusion 522. It has been unexpectedly found that pinching the portion of the porous material 504 that extend across the opening 508 forms the protrusion 522 and the protrusion 522 remains after pinching the porous material 504. In an example, the protrusion 522 may remain after pinching the porous material 504 because the pinching action plastically deforms one or more portions of the porous material 504. In an example, the protrusion 522 may remain after pinching the porous material 504 because friction between the individual layers of the porous material 504 and between the porous material 504 and the fluid impermeable barrier 502 maintains the protrusion 522. In such an example, pinching the porous material 504 may cause at least some of the portions of the porous material 504 that form the protrusion 522 to be in compression while at least some of the portions of the porous material 504 that do not form the protrusion 522 to be in tension. However, the compressive and tensile forces applied to different portions of the porous material 504 are not sufficient to overcome the kinetic friction forces and, thus, the friction maintains the protrusion 522.

Referring to FIG. 6, the fluid collection assembly 600 includes a fluid impermeable barrier 602 and at least one porous material 604 disposed in the fluid impermeable barrier 602. The porous material 604 may include a fluid permeable membrane 620 and a fluid permeable support 634. However, as previously discussed, one of the fluid permeable membrane 620 or the fluid permeable support 634 may be omitted from the fluid collection assembly 600.

The fluid collection assembly 600 includes at least one additional material 636 that is distinct from the fluid impermeable barrier 602, the fluid permeable membrane 620, and the fluid permeable support 634. The additional material 636 may be formed from at least one of a wicking material, an absorbent and/or adsorbent non-expandable material, or a fluid impermeable material (e.g., non-porous polymer or metal).

The additional material 636 is configured to affect the shape of the fluid collection assembly 600. In an embodiment, the additional material 636 is shaped and positioned to cause the porous material 604 to exhibit a protrusion 622. For example, the additional material 636 may cause the fluid collection assembly 600 to exhibit a generally tear-drop cross-sectional shape (as shown), a triangular cross-sectional shape, or any other suitable cross-sectional shape. The additional material 636 may include a rod or a plurality of distinct portions (e.g., beads). The additional material 636 may positioned within the porous material 604 to be adjacent to the opening 608 to form the protrusion 622. For example, the additional material 636 may be positioned between the fluid permeable membrane 620 and the fluid permeable support 634 which allows the additional material 636 to be easily inserted into some conventional porous materials or co-extruded, similar to the expandable material 418 of FIG. 4. Alternatively, the additional material 636 may be positioned within the fluid permeable membrane 620 or the fluid permeable support 634.

Referring to FIG. 7, the fluid collection assembly 700 includes a fluid impermeable barrier 702 and a porous material 704 disposed in the fluid impermeable barrier 702. The porous material 704 is illustrated as including a fluid permeable membrane 720 and a fluid permeable support 734 but, as previously indicated, one of the fluid permeable membrane 720 or the fluid permeable support 734 may be omitted from the porous material 704. The fluid collection assembly 700 exhibits a generally triangular cross-sectional shape that includes a protrusion 722 extending outwardly from the opening 708. For example, the fluid impermeable barrier 702 (ignoring the opening 708) and the porous material 804 may exhibit the generally triangular cross-sectional shape. The generally triangular cross-sectional shape of the fluid collection assembly 700 may exhibit the same benefits and features as the generally cross-sectional shape of the fluid collection assembly 200 of FIG. 2. In an embodiment, not shown, the fluid collection assembly 700 may include at least one additional material disposed in the porous material 704 adjacent to the opening 708 to form the protrusion 722.

The fluid collection assemblies shown in FIGS. 1A-7 are examples of female fluid collection assemblies that are configured to collect bodily fluids from females (e.g., collect urine from a female urethral opening). However, the fluid collection assemblies, systems, and methods disclosed herein may include male fluid collection assemblies shaped, sized, and otherwise configured to collect bodily fluids from males (e.g., collect urine from a male urethral opening). FIG. 8A is an isometric view of a fluid collection assembly 800, according to an embodiment. FIG. 8B is a cross-sectional view of the fluid collection assembly 800 of FIG. 8A taken along the plane 8B-8B of FIG. 8A. The fluid collection assembly 800 includes a receptacle 850 and a sheath 852. The receptacle 850 is sized, shaped, and made of a material to be coupled to skin that surrounds the male urethral opening and have the male urethral opening positioned therethrough. For example, the receptacle 850 may include an annular base 854 that defines an opening 856 in the receptacle 850. The annular base 854 is sized and shaped to be positioned around the male urethral opening (e.g., positioned around and/or over the penis) and the opening 856 may be configured to have the male urethral opening positioned therethrough. The annular basc 854 may also be sized, shaped, made of a material, or otherwise configured to be coupled (e.g., adhesively attached, such as with a hydrogel adhesive) to the skin around the male urethral opening (e.g., around the penis). In an example, the annular base 854 may exhibit the general shape or contours of the skin surface that the annular base 854 is selected to be coupled with. The annular base 854 may be flexible thereby allowing the annular base 854 to conform to any shape of the skin surface. The annular base 854 may include a laterally extending flange 855. The receptacle 850 also defines a hollowed region that is configured to receive (e.g., seal against) the sheath 852. For example, the receptacle 850 may include a longitudinally extending flange 860 that extends upwardly from the annular base 854. The longitudinally extending flange 860 may be tall enough to prevent the sheath 852 from being accidentally removed from the receptacle 850 (e.g., at least 0.25 cm tall, 1 cm tall, at least 8 cm tall, or at least 5 cm tall). The receptacle 850 is located at a proximal region 828 (with respect to a wearer) of the fluid collection assembly 800.

The sheath 852 includes (e.g., may be formed from) a fluid impermeable barrier 802 that is sized and shaped to fit into the hollowed region of the receptacle 850. For example, the sheath 852 may be generally tubular or cup-shaped, as shown. The generally tubular or cup-shaped fluid impermeable barrier 102 may at least partially define the outer surface 812 of the sheath 852. The fluid impermeable barrier 802 may be similar or identical to any of the fluid impermeable barriers disclosed herein, in one or more aspects. For example, the fluid impermeable barrier 802 may be constructed of any of the fluid impermeable materials disclosed herein. The fluid impermeable barrier 802 at least partially defines the chamber 806. For example, the inner surface 810 of the fluid impermeable barrier 802 at least partially defines the perimeter of the chamber 806. The chamber 806 may at least temporarily retain fluids therein. As shown, the fluid collection assembly 800 may include the porous material 804 therein. The porous material 804 may be similar or identical to the porous materials disclosed herein in one or more aspects. For example, the porous material 804 may include one or more of at least one expandable material 818, a fluid permeable membrane 820, or a fluid permeable support (not shown). The fluid impermeable barrier 802 may also define an opening 808 extending through the fluid impermeable barrier 802 that is configured to have a male urethral opening positioned therethrough.

The sheath 852 and fluid impermeable barrier 802 may also include at least one aperture 862 (e.g., vacuum relief hole) that allows the chamber 806 to remain substantially at atmospheric pressure. The at least one aperture 862 may be located at any point on the sheath 852, such as near or nearer the opening 856. In some examples (not shown), the aperture 862 may extend through the cap 866 or be disposed beneath the cap 866. In some examples, the fluid collection assembly 800 may not include the aperture 862, such as when a more complete seal as desired for the chamber 806.

The sheath 852 also includes at least a portion of the conduit 816 therein, such as at least partially disposed in the chamber 806. The bodily fluid may be removed from the chamber 806 via the conduit 816.

In an example, portions of the chamber 806 may be substantially empty due to the varying sizes and rigidity of the male penis. However, in some examples, the outermost regions of the chamber 806 (e.g., periphery of the interior regions of the sheath 852) may include porous material 804 (e.g., one or more of the fluid permeable membrane 820 and fluid permeable support). For example, the porous material 804 may be bonded to the inner surface 810 of the fluid impermeable barrier 802. The porous material 804 may be positioned (e.g., at the distal end of the chamber 806) to blunt a stream of urine from the male urethral opening thereby limiting splashing and/or to direct the bodily fluids to a selected region of the chamber 806. Since the chamber 806 is substantially empty (e.g., substantially all of the chamber 806 forms a reservoir), the bodily fluids are likely to pool at a gravimetrically low point of the chamber 806. The gravimetrically low point of the chamber 806 may be at an intersection of the skin of an individual and the fluid collection assembly 800, a corner formed in the sheath 852, or another suitable location depending on the orientation of the patient.

As previously discussed, the porous material 804 may include one or more of at least one expandable material 818, the fluid permeable membrane 820, or the fluid permeable support (now shown). The expandable material 818, the fluid permeable membrane 818 and the fluid permeable support may be similar or identical to any of the expandable materials, the fluid permeable membranes, or the fluid permeable supports, respectively, disclosed herein, in one or more aspects such as material make-up or wicking ability. One or more of expandable material 818, the fluid permeable membrane 820, or the fluid permeable support may be disposed between the fluid impermeable barrier 802 and a penis inserted into the chamber 806. The fluid permeable membrane 820 may be positioned between the fluid impermeable barrier 802 and a penis inserted into the chamber 806, such as between the expandable material 818 and penis of a patient, as shown. The expandable material 818 may be positioned between the fluid permeable membrane 820 and the fluid impermeable barrier 802. The inner surface 810, optionally including the end of the chamber 806 substantially opposite the opening 808, may be covered with the fluid permeable membrane 820.

As previously discussed, the expandable material 818 is configured to increase in volume when the expandable material 818 is exposed to the bodily fluids. Increasing the volume of the expandable material 818 may substantially fill at least some of the space in the chamber 806 that is not occupied by the porous material 804 and the penis. Filling such space with the expandable material 818 may reduce pooling of the bodily fluids in the chamber 806. Such pooling of the bodily fluids may cause body odor, skin degradation, and patient discomfort.

In some examples, the fluid collection assembly 800 includes a cap 866 at a distal region 826. The cap 866 defines an interior channel through which the fluids may be removed from the fluid collection assembly 800. The interior channel is in fluid communication with the chamber 806. The cap 866 may be disposed over at least a portion of a distal region 826 of one or more of the fluid impermeable barrier 802 or the porous material 804. The cap 866 may be made of a polymer, rubber, or any other fluid impermeable material. The cap 866 may be attached to one or more of the fluid impermeable barrier 802, the porous material 804, or the conduit 816. The cap 866 may have a laterally extending flange 870 and a longitudinally extending flange 872. The laterally extending flange 870 may cover at least a portion of the distal region 826 of the fluid collection assembly 800. The longitudinally extending flange 872 may laterally extend a distance from the sheath 852. The longitudinally extending flange 872 is sized and configured to receive and fluidly seal against the conduit 816, such as within the interior channel. The conduit 816 may extend a distance within or through the cap 866, such as to the porous material 804, through the porous material 804, or to a point set-off from the porous material 804. In the latter example, as depicted in FIG. 8B, the interior channel of the cap 866 may define a fluid reservoir 824 therein.

The reservoir 824 is an unoccupied portion of fluid collection assembly 800 such as in the cap 866 and is void of other material. In some examples, the reservoir 824 is defined at least partially by the porous material 804 and the cap 866. During use, the fluids that are in the chamber 806 may flow through the porous material 804 to the reservoir 824. The reservoir 824 may store at least some of the fluids therein and/or position the fluids for removal by the conduit 816. In some examples, at least a portion of the porous material 804 may extend continuously between at least a portion of the opening of the interior channel and chamber 806 to wick any fluid from the opening directly to the reservoir 824.

In some examples (not shown), the fluid impermeable barrier 802 may be disposed on or over the cap 866, such as enclosing the cap 866 within the chamber 806.

In some examples, the sheath 852 may include at least a portion of the conduit 816 therein, such as at least partially disposed in the chamber 806. For example, the conduit 816 may extend from the sheath 852 to a region at least proximate to the opening 856. The inlet of the conduit 816 may be positioned adjacent to the annular base 854. The inlet of the conduit 816 may be positioned to be adjacent or proximate to the gravimetrically low point of the chamber 806, such as adjacent to the annular base 854. For example, the inlet may be co-extensive with or offset from the opening 856. In examples, the inlet may be positioned adjacent to the distal region 826 of the sheath 852 (e.g., substantially opposite the opening).

The proximal region 828 may be disposed near or on the skin around the male urethral opening (e.g., around the penis) and the inlet of the conduit 816 may be positioned in the proximal region 842. The outlet of the conduit 816 may be directly or indirectly coupled to a vacuum source. Accordingly, fluid may be removed from the proximal region 842 of the chamber 806 via the conduit 816.

The receptacle 850, the sheath 852, the cap 866, and the conduit 816 may be attached together using any suitable method. For example, at least two of the receptacle 850, the sheath 852, the cap 866, or the conduit 816 may be attached together using at least one of an interference fit, an adhesive, stitching, welding (e.g., ultrasonic welding), tape, any other suitable method, or combinations thereof.

In some examples (not shown), the fluid collection assembly 800 may have a one piece design, with one or more of the sheath 852, the receptacle 850, and the cap 866 being a single, integrally formed piece.

Also as shown, the conduit 816 may be at least partially disposed with the chamber 806 of a fluid collection assembly 800. The conduit 816 may extend from the distal region 826 to the proximal region 828. For example, the conduit 816 may extend through the cap 866 to a point adjacent to the receptacle 850. The conduit 816 is sized and positioned to be coupled to a fluid storage container or the vacuum source (FIG. 9). An outlet of the conduit 816 may be operably coupled to the vacuum source, directly or indirectly. The inlet of the conduit 816 may be positioned within the chamber 806 such as at a location expected to be at the gravimetrically low point of the fluid collection assembly during use. By positioning the inlet in a location expected to be at the gravimetrically low point of the fluid collection assembly when worn by the user, fluids introduced into the chamber 806 may be removed via the conduit 816 to prevent pooling or stagnation of the fluid within the chamber 806.

In some examples, the vacuum source may be remotely located from the fluid collection assembly 800. In such examples, the conduit 816 may be fluidly connected to the fluid storage container, which may be disposed between the vacuum source and the fluid collection assembly 800.

During operation, a male using the fluid collection assembly 800 may discharge the bodily fluids (e.g., urine) into the chamber 806. The bodily fluids may pool or otherwise be collected in the chamber 806. At least some of the bodily fluids may be pulled through the interior of the conduit 816 via the inlet. The bodily fluids may be drawn out of the fluid collection assembly 800 via the vacuum/suction provided by the vacuum source. During operation, the aperture 862 may substantially maintain the pressure in the chamber 806 at atmospheric pressure even though fluid is introduced into and subsequently removed from the chamber 806.

Other examples of male fluid collection assemblies that may include at least one expandable material are disclosed in International Application No. WO 2021/016026 filed on Jul. 16, 2020, U.S. patent application Ser. No. 16/433,773 filed on Apr. 3, 2020, and International Application No. PCT/2021/039866 filed on Jun. 30, 2021, the disclosure of each of which is incorporated herein, in its entirety, by this reference.

FIG. 9 is a block diagram of a system 901 for fluid collection, according to an embodiment. The system 901 includes a fluid collection assembly 900, a fluid storage container 907, and a vacuum source 909. The fluid collection assembly 900, the fluid storage container 907, and the vacuum source 909 may be fluidly coupled to each other via one or more conduits 916. For example, fluid collection assembly 900 may be operably coupled to one or more of the fluid storage container 907 or the vacuum source 909 via the conduit 916. Fluid (e.g., urine or other bodily fluids) collected in the fluid collection assembly 900 may be removed from the fluid collection assembly 900 via the conduit 916 which protrudes into the fluid collection assembly 900. For example, an inlet of the conduit 916 may extend into the fluid collection assembly 900, such as to a reservoir therein. The outlet of the conduit 916 may extend into the fluid collection assembly 900 or the vacuum source 909. Suction force may be introduced into the chamber of the fluid collection assembly 900 via the inlet of the conduit 916 responsive to suction (e.g., vacuum) force applied at the outlet of the conduit 916.

The suction force may be applied to the outlet of the conduit 916 by the vacuum source 909 either directly or indirectly. The suction force may be applied indirectly via the fluid storage container 907. For example, the outlet of the conduit 916 may be disposed within the fluid storage container 907 and an additional conduit 916 may extend from the fluid storage container 907 to the vacuum source 909. Accordingly, the vacuum source 909 may apply suction to the fluid collection assembly 900 via the fluid storage container 907. The suction force may be applied directly via the vacuum source 909. For example, the outlet of the conduit 916 may be disposed within the vacuum source 909. An additional conduit 916 may extend from the vacuum source 909 to a point outside of the fluid collection assembly 900, such as to the fluid storage container 907. In such examples, the vacuum source 909 may be disposed between the fluid collection assembly 900 and the fluid storage container 907.

The fluid collection assembly 900 may be similar or identical to any of the fluid collection assemblies disclosed herein (e.g., 100 and 300-1300) in one or more aspects. The fluid collection assembly 900 may be shaped and sized to be positioned adjacent to a female urethral opening or have a male urethral opening positioned therethrough (e.g., receive a penis therein). For example, the fluid collection assembly 900 may include a fluid impermeable barrier at least partially defining a chamber (e.g., interior region) of the fluid collection assembly 900. The fluid impermeable barrier also defines an opening extending therethrough from the external environment. The opening may be positioned adjacent to a female urethral opening or have a male urethral opening positioned therethrough. The fluid collection assembly 900 may include a fluid permeable membrane disposed within the fluid impermeable barrier. The fluid collection assembly 900 may include wicking material disposed in the chamber such as one or more of a fluid permeable support and a fluid permeable membrane. The fluid collection assembly 900 includes the shape memory material on or incorporated in one or more components thereof. The shape memory material is sized, shaped, and positioned to retain a selected geometric configuration as disclosed herein. The conduit 916 may extend into the fluid collection assembly 900 at a first end (e.g., proximal) region, through one or more of the fluid impermeable barrier, fluid permeable membrane, or the fluid permeable support to a second end (e.g., distal) region of the fluid collection assembly 900. The conduit 916 includes an inlet and an outlet, the outlet being fluidly coupled to the fluid storage container and the inlet being positioned in a portion of the chamber selected to be at a gravimetrically low point of the fluid collection assembly when worn.

The fluid storage container 907 is sized and shaped to retain a fluid therein. The fluid storage container 907 may include a bag (e.g., drainage bag), a bottle or cup (e.g., collection jar), or any other enclosed container for storing bodily fluid(s) such as urine. In some examples, the conduit 916 may extend from the fluid collection assembly 900 and attach to the fluid storage container 907 at a first point therein. An additional conduit 916 may attach to the fluid storage container 907 at a second point thereon and may extend and attach to the vacuum source 909. Accordingly, a vacuum (e.g., suction) may be drawn through fluid collection assembly 900 via the fluid storage container 907. Fluid, such as urine, may be drained from the fluid collection assembly 900 using the vacuum source 909.

The vacuum source 909 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 909 may provide a vacuum or suction to remove fluid from the fluid collection assembly 900. In some examples, the vacuum source 909 may be powered by one or more of a power cord (e.g., connected to a power socket), one or more batteries, or even manual power (e.g., a hand operated vacuum pump). In some examples, the vacuum source 909 may be sized and shaped to fit outside of, on, or within the fluid collection assembly 900. For example, the vacuum source 909 may include one or more miniaturized pumps or one or more micro pumps. The vacuum sources 909 disclosed herein may include one or more of a switch, a button, a plug, a remote, or any other device suitable to activate the vacuum source 909.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

We claim:

1. A fluid collection assembly, comprising:
a fluid impermeable barrier at least defining a chamber, at least one opening, and a fluid outlet; and
at least one porous material disposed in the chamber, the at least one porous material including at least one of:
a protrusion extending outwardly from the at least one opening, the protrusion exhibiting a shape that does not correspond to a shape of the fluid impermeable barrier and a shape of a majority of the porous material, wherein the at least one porous material includes the protrusion before the at least one porous material is exposed to one or more bodily fluids; or
a fluid permeable membrane, a fluid permeable support, and at least one expandable material that expands when exposed to the one or more bodily fluids, the fluid permeable membrane extending across the at least one opening, the fluid permeable membrane positioned between the at least one expandable material and at least a portion of the fluid impermeable barrier, the at least one expandable material positioned between at least a portion of the fluid permeable membrane and the fluid permeable support, wherein an expanded portion of the at least one porous material exhibits a shape that does not correspond to a shape of the fluid impermeable barrier and a shape of the majority of a porous material when the at least one expandable material is expanded.
